(19) United States
(12) Patent Application Publication
(10) Pub. No.: US 2026/0151552 A1
(43) Pub. Date: Jun. 4, 2026

(54) DEVICES FOR DIRECTED INSTILLATION OR IRRIGATION OF FLUID DURING WOUND THERAPY

(71) Applicant: Fistula Solution Corporation, Scandia, MN (US)

(72) Inventors: Andrew T. Obst, Scandia, MN (US); Mary Anne R. Obst, Scandia, MN (US)

(21) Appl. No.: 19/404,841

(22) Filed: Dec. 1, 2025

Related U.S. Application Data
(60) Provisional application No. 63/726,884, filed on Dec. 2, 2024.

(51) Int. Cl. A61M 1/00; A61F 13/05

(52) U.S. Cl. CPC A61M 1/92; A61F 13/05; A61M 1/912

(57) ABSTRACT

The present disclosure describes techniques for providing directed instillation or irrigation of fluid during wound therapy to deliver cleansing fluid precisely onto a wound to improve wound cleansing. Directed instillation or irrigation of fluid onto wounds using techniques of the present disclosure can accelerate wound healing and provide improved patient outcomes. One example of the present disclosure is a device for providing directed fluid irrigation to a wound. The device can include an irrigation tube extending between a first end and a second end, with the second end having a perforated section or a non-perforated closed section. The device can further include a foam structure arranged around the perforated section, with the foam structure being configured to dispense fluid received from the perforated section uniformly into a wound for cleansing. The perforated section can include a plurality of perforations or a plurality of perforated irrigation tubes in examples.

### RELATED APPLICATIONS

This application claims the benefit of U.S. Provisional Application 63/726,884, filed Dec. 2, 2024, which is incorporated herein by reference in its entirety.

### TECHNICAL FIELD

The present disclosure relates generally to devices and methods that provide periodic fluid irrigation to patient wounds during therapy. More specifically, the present disclosure relates to devices and methods that provide directed instillation or irrigation of fluid during wound therapy to deliver cleansing fluid precisely onto a wound to improve cleansing of deep, tunneling, undermined, and uneven wound beds which can accelerate wound healing.

### BACKGROUND

Wound therapy has evolved significantly over recent decades. One major advancement is the combination of negative pressure wound therapy (NPWT) with the instillation or irrigation of therapeutic fluids. This combined approach, often referred to as negative pressure wound therapy with instillation (NPWTi), has been discussed in various patent documents including U.S. Patent Publ. No. 2021/0370043; EP Patent No. 3,313,469; U.S. Pat. No. 9,526,817; and CA Patent No. 2,881,742, for example. These documents highlight the integration of fluid instillation with NPWT to enhance wound care outcomes.

The primary advantage of NPWTi is the ability to intermittently cleanse wounds during therapy without removing the wound dressing. This intermittent cleansing reduces infection risks and promotes tissue regeneration, leading to improved healing rates. By delivering therapeutic fluids directly to the wound site, NPWTi can effectively remove exudate, debris, and infectious materials from the wound, thereby creating an optimal environment for wound healing.

Despite the advancements offered by NPWTi, conventional approaches to fluid instillation have certain limitations. Traditional NPWTi techniques often rely on gravity to draw fluid through the wound dressing which can result in uneven fluid distribution. This is particularly problematic in wounds with complex geometries, such as deep, tunneling, and undermining wounds, or wounds with uneven beds. In such cases, the gravitational flow may not adequately reach all areas of the wound, leading to suboptimal cleansing and potential delays in healing. Furthermore, the reliance on gravity-based fluid delivery methods and devices can complicate the treatment of patients with wounds in non-standard positions or those who are bedridden, as the orientation of the wound relative to the fluid source can affect the efficacy of the instillation process.

In light of these limitations, there is a need for improved wound therapy techniques for delivering cleansing fluids precisely and uniformly across a wound bed, regardless of the shape of the wound or the position of the patient. Such techniques would not only enhance the effectiveness of wound cleansing, but also accelerate the healing process by ensuring that all areas of a wound receive adequate treatment.

### SUMMARY

The present disclosure addresses the limitations of conventional approaches to fluid irrigation by providing techniques that enable directed instillation or irrigation of fluids during wound therapy to deliver cleansing fluid precisely onto a wound. This improves wound cleansing particularly in deep, tunneling, and undermined wounds, and wounds with uneven beds. Techniques of the present disclosure also improve wound cleansing for patients with wounds in non-standard positions or patients that are bedridden. Such techniques can also accelerate healing by providing fluid irrigation to all sections of a wound to ensure adequate cleansing treatment of the entire wound area.

Examples of key, advantageous features of the present disclosure include providing a flexible, small diameter medical-grade irrigation tube couplable to various fluid injection sources (e.g., NPWT pumps, IV pumps, syringes, gravity bags, etc.); providing an irrigation tube extending between a first end and a second end, with the second end having a plurality of perforations or apertures dispersed along a length to facilitate fluid dispersion out of the tube (e.g., perforations or apertures dispersed along a length of approximately 10 to 20 centimeters (approximately 3.93 to 7.87 inches)); providing an irrigation tube with an optional second perforated end embedded within a hydrophobic, open-celled foam to ensure uniform fluid distribution out of the tube and onto a wound; providing customizability to the irrigation tube partially embedded within the hydrophobic, open-celled foam (e.g., the foam and the irrigation tube can be trimmed to fit wound-specific geometries, including deep tunnels, irregular surfaces, and non-standard wound positions).

The techniques of the present disclosure are advantageous because they provide precise fluid delivery to wounds which ensures effective cleansing of deep or uneven wound beds among other wound types. Other advantages include uniform fluid distribution within a wound by using one or more irrigation tubes couplable to or embedded within hydrophobic foam, enhanced adaptability by providing customizable tube and foam designs to accommodate various wound shapes and sizes and various patient characteristics (e.g., non-standard wound positions, bedridden or difficult to reach patients), and improved healing enabled through improved cleansing which reduces infection risk and accelerates patient recovery.

Examples of the present disclosure describe a device for providing directed fluid irrigation to a wound. The device can include an irrigation tube extending between a first end and a second end. The device can further include a foam structure proximate the second end, with the foam structure being configured to dispense fluid received from the irrigation tube uniformly into and around a wound for cleansing. The second end can comprise one of a perforated section having a plurality of perforations arranged around an outer circumference of the second end, a section having a plurality of perforated or non-perforated irrigation tubes extending outward from the irrigation tube (e.g., a plurality of perforated irrigation tubes, a plurality of non-perforated irrigation tubes, or a mix of perforated and non-perforated irrigation tubes), and a closed section terminating in the foam structure.

Examples of the present disclosure describe a system for providing directed fluid irrigation to a wound. The system can include an irrigation device having an irrigation tube extending between a first end and a second end, and a foam structure proximate the second end. The system can further include an injection fluid source couplable to the first end of the irrigation tube, with the injection fluid source being configured to provide fluid to the irrigation tube between the first end and the second end. The foam structure can be configured to dispense fluid received from the irrigation tube into and around a wound for cleansing.

Examples of the present disclosure describe a method for providing directed fluid instillation or irrigation to a wound. The method can include positioning an irrigation device proximate a wound, with the irrigation device including an irrigation tube extending between a first end and a second end, and a foam structure proximate the second end. The method can further include injecting cleansing fluid through the irrigation tube to the foam structure and outward from the foam structure to the wound for cleansing. The method can further include applying negative pressure to the irrigation device to remove fluid and contaminants from the wound.

### BRIEF DESCRIPTION OF THE DRAWINGS

Subject matter hereof may be more completely understood in consideration of the following detailed description of various examples in connection with the accompanying figures, in which:

FIG. 1A depicts a first wound irrigation system for providing directed fluid instillation or irrigation to a wound during therapy, according to examples of the present disclosure;

FIG. 1B depicts an irrigation tube having a perforated section including a plurality of perforations or apertures, according to examples of the present disclosure;

FIG. 1C depicts cross-sectional profiles of a foam structure for use with an irrigation tube, according to examples of the present disclosure;

FIG. 2 depicts the first wound irrigation system of FIG. 1A in operation with a patient wound, according to examples of the present disclosure;

FIG. 3A depicts a second wound irrigation system for providing directed fluid instillation or irrigation to a wound during therapy, according to examples of the present disclosure;

FIG. 3B depicts an irrigation tube having a perforated section including a plurality of perforated irrigation tubes extending outward, according to examples of the present disclosure;

FIG. 4 depicts the second wound irrigation system of FIG. 3A in operation with a patient wound, according to examples of the present disclosure;

FIG. 5A depicts an irrigation tube having a non-perforated section including a closed section with an opening at a distal end, according to examples of the present disclosure; and

FIG. 5B depicts an irrigation tube having a non-perforated section including a plurality of non-perforated irrigation tubes extending outward, according to examples of the present disclosure.

While various examples are amenable to various modifications and alternative forms, specifics thereof have been shown by way of example in the drawings and will be described in detail. It should be understood, however, that the intention is not to limit the claimed inventions to the particular examples described. On the contrary, the intention is to cover all modifications, equivalents, and alternatives falling within the spirit and scope of the subject matter as defined by the claims.

### DETAILED DESCRIPTION OF THE DRAWINGS

The present disclosure describes examples of devices, methods, systems, and techniques for directed instillation or irrigation of fluid during wound therapy. Examples of the present disclosure are designed to deliver cleansing fluid precisely onto a wound bed during wound therapy or treatment. This provides improved cleansing of deep, tunneling, undermined, and uneven wound beds, which can accelerate wound healing. Devices of the present disclosure may also be designed to be cut to size or overlapped as needed to fit specific patient wound shapes and sizes. In general, the present disclosure significantly enhances wound therapy by enabling targeted fluid delivery and effective cleansing of various wound types, thereby addressing certain limitations of conventional NPWTi techniques and other conventional wound therapy systems.

Unless otherwise indicated, all numbers expressing feature sizes, amounts, ranges, and physical properties are to be understood as being modified by the term “about.” Accordingly, unless indicated to the contrary, the numerical parameters set forth are approximations that can vary depending on the desired properties sought by those skilled in the art utilizing the teachings disclosed herein. As used herein, the singular forms “a,” “an,” and “the” encompass examples with plural referents, unless the context clearly dictates otherwise. Similarly, the term “or” is generally employed in its inclusive sense (“and/or”) unless the context specifies otherwise. The terms “top,” “upper,” “bottom,” or “lower” are used to describe the relative position of two or more components within the device or a patient's skin. The terms “first” and “second” are used to describe identical or substantially identical features and may be used interchangeably (e.g., “first end” may refer to “second end” and vice versa).

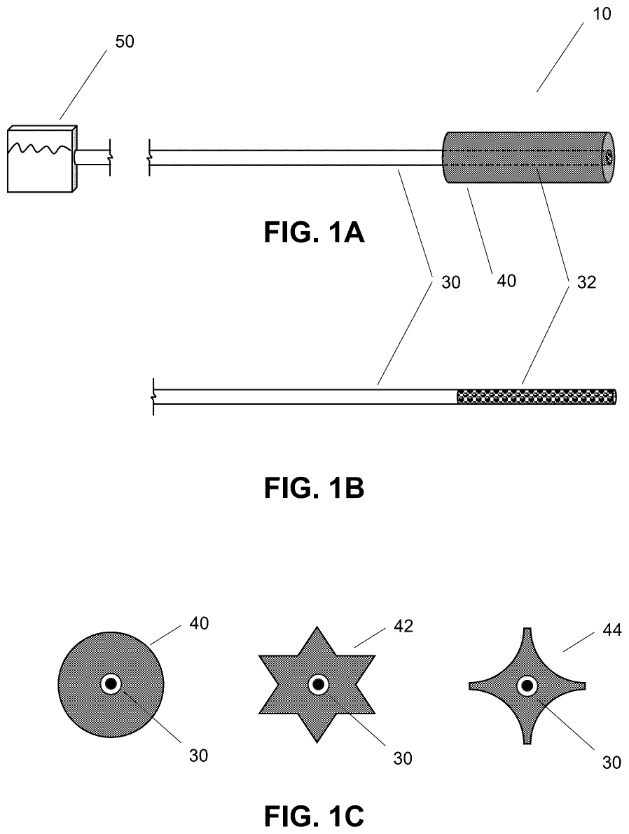

Referring generally to FIGS. 1A-1C, a first wound irrigation system 10 providing directed fluid instillation or irrigation to a wound during therapy is depicted according to examples of the present disclosure. First system 10 can treat a variety of wounds including deep, tunneling, and undermined wounds, and uneven wound beds, among other wound types.

First system 10 may comprise a flexible medical-grade irrigation tube 30 extending from a first end to a second end, a foam structure 40 couplable around the second end of tube 30, and an injection fluid source 50 couplable to the first end of tube 30 and configured to provide irrigation fluid to tube 30 for cleansing of wounds. Irrigation tube 30 and foam structure 40 in combination may be characterized as a “device” or an “irrigation device” suitable for use with any injection fluid source 50 capable of being coupled to medical-grade tubing. In other examples, the components of first system 10 may be provided individually or provided as a “kit” to be assembled by a medical practitioner or a patient.

Irrigation tube 30 may have a cylindrical geometry extending between the first and second ends. In other examples, irrigation tube 30 may have other geometries such as conical, cubical, pyramidal, or other suitable polygonal geometries. Irrigation tube 30 can define any suitable length between the first and second ends necessary to enable fluid instillation or irrigation to a wound during operation of first system 10. In examples, irrigation tube 30 can define a length of approximately 0.5 to 3 meters (approximately 20 to 120 inches), though other lengths less than or greater than this range are contemplated by the present disclosure. Irrigation tube 30 can be characterized as a thin-walled structure or a thick-walled structure, with an outer diameter in the range of approximately 0.3175 to 5.08 centimeters (approximately ⅛ to 2 inches) and an inner diameter slightly or substantially less than the outer diameter. Other outer diameters less than or greater than this range are contemplated by the present disclosure.

When irrigation tube 30 comprises a thin-walled structure, the inner diameter may be slightly less than the outer diameter (e.g., approximately 1 to 15% difference in diameter value). When irrigation tube 30 comprises a thick-walled structure, the inner diameter may be substantially less than the outer diameter (e.g., greater than approximately 15% difference in diameter value). The use of a thin or thick-walled irrigation tube 30 is generally dictated by the type of wound to be cleansed (e.g., smaller or larger wounds, non-standard positioned wounds, patient characteristics, etc.). Irrigation tube 30 may comprise any medical-grade material suitable for fluid dispersal therein, for example metals, ceramics, composites, polymers, or combinations thereof. Suitable polymer materials include polyvinyl chloride (PVC), polyurethane, silicone, fluorinated ethylene propylene (FEP), perfluoroalkoxy (PFA), polyvinylidene fluoride (PVDF), polytetrafluoroethylene (PTFE), and shape memory polymers (SMPs).

The second end of irrigation tube 30 may define a perforated section 32 comprising a plurality of perforations or apertures as depicted particularly in FIG. 1B. The plurality of perforations or apertures defining the perforated section 32 may be dispersed regularly or irregularly along a length extending from the second end. In examples, the plurality of perforations or apertures may be defined along a length of approximately 10 to 20 centimeters (approximately 4 to 8 inches) starting from the second end, though other perforation lengths less than or greater than this range are contemplated by the present disclosure. The plurality of perforations or apertures may be arranged around the entire outer cylindrical circumference or other outer circumferential geometry of irrigation tube 30 at the second end, or arranged around only a portion of the geometry (e.g., 50% of the outer circumferential geometry). The plurality of perforations or apertures may be through holes defined between the outer and inner surfaces of irrigation tube 30. This enables fluid communication between the plurality of perforations or apertures and any fluid flowing through irrigation tube 30.

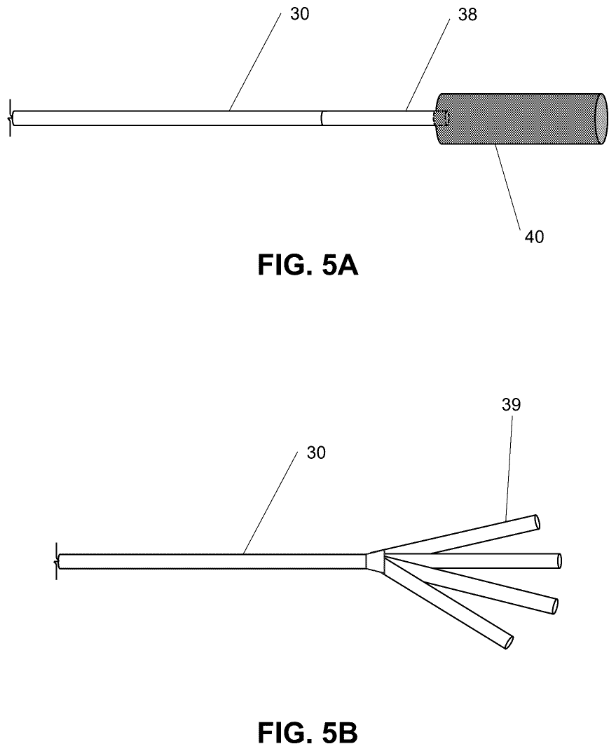

In other examples, the second end of the irrigation tube 30 may define a non-perforated closed section 38 that terminates or ends in the foam structure 40, as depicted in FIG. 5A (e.g., foam structure 40 can be positioned proximate the second end). Said another way, the second end may be closed or solid around the entire outer circumferential geometry of irrigation tube 30 at the second end. The second end may include an opening or fluid exit port that terminates or ends in the foam structure 40. This enables fluid to be directly transmitted to or directly received by the foam structure 40 via the irrigation tube 30 through the opening or fluid exit port. This configuration provides directed fluid delivery or dispersal exclusively through the foam structure 40. In such examples, the second end of irrigation tube 30 may extend entirely or partially into foam structure 40 for fluid dispersal therein (e.g., the second end and the closed section 38 may terminate or end at the same point where foam structure 40 starts). This configuration is particularly advantageous for wounds requiring precise fluid delivery at a single point or localized area.

The first end of irrigation tube 30 may be couplable to the injection fluid source 50. This enables fluid flow from the injection fluid source 50 and into the irrigation tube 30 between the first and second ends. Accordingly, fluid can be dispersed or dispensed out of the plurality of perforations or apertures during fluid communication or coupling between injection fluid source 50 and irrigation tube 30. In examples, when the perforated section 32 is arranged around the entire outer circumference of irrigation tube 30, fluid can be dispersed out of the plurality of perforations or apertures in all directions to provide uniform fluid dispersion and cleansing to a wound.

As depicted in FIGS. 1A and 1C, foam structure 40 may be couplable around the perforated section 32 defined at the second end of irrigation tube 30. In other words, irrigation tube 30 may be removably or fixedly embedded within foam structure 40 at the perforated section 32. In other examples where second end defines a closed section 38, such as those depicted in FIG. 5A, the closed section 38 may extend entirely or partially into foam structure 40 and terminate therein (e.g., the closed section 38 can be proximate the foam structure 40). An opening or fluid exit port of the closed section 38 may be in fluid communication with foam structure 40 to dispense fluid from the irrigation tube 30 to the foam structure 40 for wound cleansing.

Foam structure 40 may comprise a hydrophobic, open-celled foam having an outer diameter in the range of approximately 0.635 to 12.7 centimeters (approximately ¼ to 5 inches), and an inner diameter approximately equal to the outer diameter of irrigation tube 30. Other outer and inner diameter combinations less than or greater than these values are contemplated by the present disclosure for foam structure 40 (e.g., foam structure 40 may have a 10% or 50% difference, or other difference, between outer and inner diameters, depending on the type of wound to be cleansed, the needs of the patient, and the dimensions of the irrigation tube 30 that is used). Foam structure 40 may comprise any medical-grade foam material suitable for fluid dispersal therethrough, for example polyurethane foam, melamine foam, metal foams made by Duocel®, beaded or crosslinked polyethylene foams, and expanded polystyrene foam, for example.

Foam structure 40 may have a cylindrical geometry extending between a first end and a second end in certain examples. In other examples, foam structure 40 may have a star-shaped geometry 42 or a polygonal prism winged-shaped geometry 44 as depicted in FIG. 1C. Other polygonal geometries are contemplated by the present disclosure for foam structure 40, including cubical, pyramidal, pentagonal, hexagonal, or other suitable polygon shapes. The second end of foam structure 40 may be aligned or substantially aligned with the second end of irrigation tube 30 when tube 30 is coupled to or embedded within structure 40.

Foam structure 40 may have a length equivalent or substantially equivalent to the length of perforated section 32. In examples, foam structure 40 and perforated section 32 may each have a length of approximately 10 to 20 centimeters (approximately 4 to 8 inches). In other examples, foam structure 40 may have a length less than or greater than a length of the perforated section 32 defined by irrigation tube 30. In other examples where second end defines a closed section 38, such as those depicted in FIG. 5A, the closed section 38 generally has a length less than a length of foam structure 40. However, the present disclosure contemplates examples where the length of the closed section 38 is greater than the length of foam structure 40. In examples, foam structure 40 and irrigation tube 30 can be trimmed or cut to length to fit wound-specific geometries including deep wound tunnels and irregular wound surfaces. The trimming or cutting to length may occur immediately prior to wound therapy or treatment, for example at the bedside of a patient. This customizability enables use of first system 10 and the associated components with a wide range of wounds, patients, and therapy or treatment settings.

When used with irrigation tube 30, foam structure 40 can provide uniform fluid distribution out of the plurality of perforations or apertures defined by the perforated section 32 and onto a patient wound in all areas (e.g., uniform or substantially uniform along a wound tunnel, channel, or opening). In other examples, foam structure 40 can receive fluid from an opening defined at a distal point of the second end of irrigation tube 30 when the second end includes a non-perforated closed section or a perforated section. In examples, the hydrophobic, open-celled foam of foam structure 40 enables even or substantially even fluid dispersal throughout a wound. This provides improved wound cleansing especially for deep, tunneling, undermined, and uneven wound beds, and for patients with wounds in non-standard positions or those who are bedridden. More precise cleansing of wounds can accelerate wound healing which can improve outcomes for patients undergoing wound therapies or treatments.

Injection fluid source 50 may comprise any suitable fluid supply including an external or internal fluid reservoir removably couplable to the irrigation tube 30. Injection fluid source 50 may comprise electrical or mechanical circuitry or components configured to start, stop, pause, and resume fluid flow between the fluid source 50 and irrigation tube 30. In examples, injection fluid source 50 may comprise electrical or mechanical circuitry or components configured to control the rate of fluid flow into the irrigation tube 30. In examples, injection fluid source 50 may be configured to inject cleansing fluid intermittently or continuously into the insertion tube 30 for controlled fluid dispersal onto a wound. Rate-controlled fluid dispersal can be particularly useful for wounds that require more specialized cleansing and treatment compared to standard fluid dispersal at a continuous, non-changing rate. In examples, injection fluid source 50 may comprise a negative pressure wound therapy (NPWT) pump, an intravenous (IV) pump, a syringe, a gravity bag, or combinations thereof for delivering fluid to irrigation tube 30. Injection fluid source 50 may comprise a storage reservoir configured to hold a quantity of fluid. This is useful for situations when first system 10 is transported for wound therapy or treatment, for example in an ambulance during an emergency event or in a remote location away from a reliable source of continuous fluid.

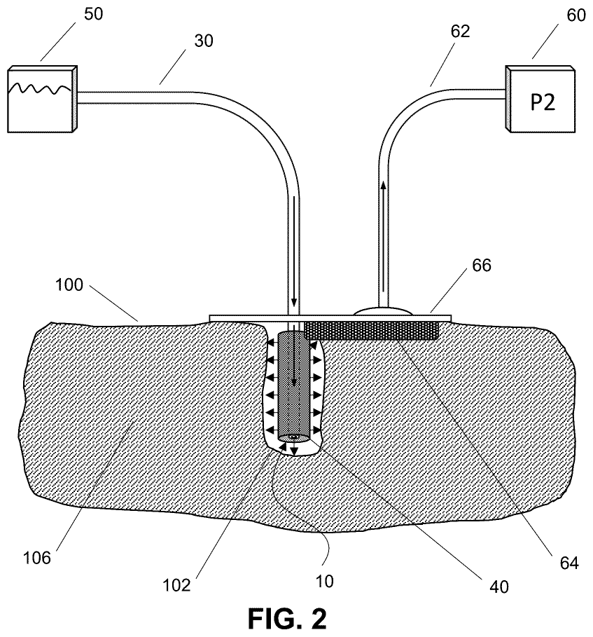

Referring now to FIG. 2, operation of the first wound irrigation system 10 in a deep tunneling wound 102 of a patient is depicted. The second end of the irrigation tube 30 can be cut to length and sized to fill the wound tunnel from the base of the wound 102 to a patient skin surface 100. In use, an insertion tube 30 coupled to or embedded within a foam structure 40 at a second end can be inserted into the wound tunnel. The first end of the insertion tube 30 can be couplable or placed in fluid communication with an injection fluid source 50 configured to dispense or disperse fluid through the insertion tube 30. A negative pressure wound therapy system can then be applied over the “irrigation device” comprising the irrigation tube 30 and foam structure 40 in combination. The negative pressure wound therapy system generally includes a layer of open-celled foam or other wound filler 64 sealed with a film or drape 66, and couplable via a negative pressure vacuum tube and wound dressing connector 62 to a negative pressure source 60. In examples, negative pressure source 60 may comprise a negative pressure pump or any other suitable vacuum source with a pressure P₂ less than atmospheric pressure P₁. In other examples, the wound irrigation system 10 may be used without a negative pressure source and the wound filler 64 will act as a passive wound cover dressing.

After placement, the injection fluid source 50 can inject fluid through the irrigation device 30, 40 intermittently, continuously, or at a controlled rate into a patient wound. Fluid travels from the injection fluid source 50 to the first end of the irrigation tube 30, followed by traveling to the second end of the irrigation tube 30 and being dispersed out of the plurality of perforations or apertures defined at the perforated section 32. In other examples, fluid can be dispersed out of an opening defined at a distal point of the second end when the second end defines a non-perforated closed section or a perforated section (e.g., an opening or channel positioned between the first and second ends of the irrigation tube 30).

The fluid is dispersed into the foam structure 40 that the irrigation tube 30 is embedded within, followed by dispersal out of the foam structure 40 and onto and around the wound tunnel defining the wound 102, using an even or substantially even dispersion. Subsequently, the negative pressure source 60 can draw the fluid out of the wound bed 102 through the open-celled foam or other wound filler 64. The negative pressure vacuum tube 62 can remove the cleansing fluid from the patient, thereby effectively removing exudate, debris, and infectious materials from the wound to promote improved healing.

The entire operation of first wound irrigation system 10, or alternatively the “irrigation device” comprising the irrigation tube 30 and the foam structure 40, is depicted in FIG. 2. Specifically, fluid is directed from the injection fluid source 50 through the irrigation tube 30 and dispersed out of the foam structure 40 onto the wound 102 defined within a patient skin surface 100. The dispersed fluid can be drawn out of the wound 102 using the negative pressure source 60 in combination with the negative pressure vacuum tube 62 and the open-celled foam or other wound filler 64. Accordingly, first system 10 provides directed instillation or irrigation of fluid during wound therapy to deliver cleansing fluid precisely onto and around a wound to improve cleansing of deep, tunneling, undermined, and uneven wound beds. The removal of contaminants and other debris from the wound accelerates wound healing for the patient.

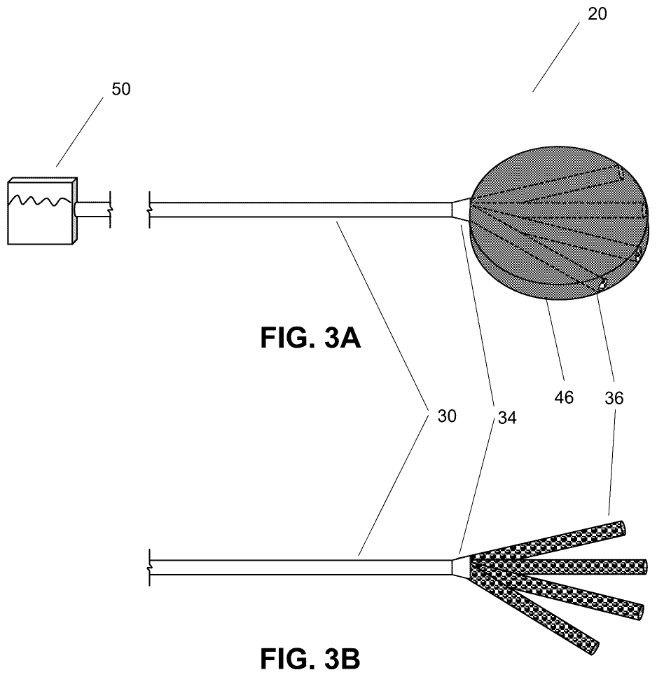

Referring now generally to FIGS. 3A-3B, a second wound irrigation system 20 providing directed fluid instillation or irrigation to a wound during therapy is depicted according to examples of the present disclosure. Second system 20 can treat a variety of wounds including deep, tunneling, and undermined wounds, and uneven wound beds, among other wound types. Second system 20 is particularly suitable for larger wounds requiring an expanded fluid dispersion area as illustrated in the figures. Second system 20 is also particularly suitable for uneven wound beds with creases or ridges.

Second system 20 has certain similarities to first system 10. For simplicity, certain descriptions of common components are not repeated in the following. Like reference numerals and feature names may designate like feature names throughout that are corresponding or analogous. For example, as with first system 10, second system 20 may include an irrigation tube 30 and an injection fluid source 50. Irrigation tube 30 and injection fluid source 50 of second system 20 are generally functionally identical to the same components used with first system 10. However, various similarities and differences between first and second systems 10, 20 will be highlighted in the forthcoming description.

Second system 20 may comprise an irrigation tube 30 extending from a first end to a second end, with the second end being couplable to a manifold 34 that distributes fluid into a plurality of perforated irrigation tubes 36 extending outward from the irrigation tube 30. The plurality of perforated irrigation tubes 36 can be couplable to or embedded within a foam structure 46. Foam structure 46 can be characterized as a thin, flat pad of hydrophobic open-celled foam (e.g., foam structure 46 can have a flat sheet or disk-like geometry). Similar to foam structure 40 of first system 10, foam structure 46 can disperse fluid received from an injection fluid source 50 into, around, and across a wound tunnel, including wounds that are larger and wounds that have uneven beds with creases or ridges. Foam structure 46 and the plurality of perforated irrigation tubes 36 can be cut to size to match the specific shape and contours of a wound. Both components can then be placed directly onto a bed of a wound for precise fluid dispersal therein.

In examples, irrigation tube 30, manifold 34, the plurality of perforated irrigation tubes 36, and foam structure 46 in combination may be characterized as a “device” or an “irrigation device” suitable for use with any injection fluid source 50. The “irrigation device” may comprise other components or features necessary to effect fluid dispersal onto a wound using the techniques described herein. In examples, the irrigation device can include a plurality of non-perforated irrigation tubes 39, as depicted in FIG. 5B, instead of perforated tubes 36. The plurality of non-perforated tubes can terminate or end in foam structure 46, with each non-perforated tube having an opening or channel that fluid can be dispensed out of. In other examples, the irrigation device can include a mix of non-perforated irrigation tubes 39 and perforated irrigation tubes 36 for a combined fluid dispersal approach within foam structure 46.

The plurality of perforated irrigation tubes 36 (or non-perforated, closed irrigation tubes 39 as depicted in FIG. 5B) may be equidistant from each other or at varying distances as needed. The plurality of perforated irrigation tubes 36 may each define a plurality of perforations or apertures arranged entirely or partially around an outer circumferential surface. Similar to perforated section 32 of first system 10, the plurality of perforations or apertures may be through holes defined between the outer and inner surfaces of each perforated irrigation tube 36. This enables fluid communication between the plurality of perforations or apertures and any fluid flowing from the irrigation tube 30 to the plurality of perforated irrigation tubes 36. In examples, the plurality of perforated irrigation tubes 36 can be characterized as a perforated section of irrigation tube 30 (e.g., irrigation tube 30 can define a non-perforated section extending between first and second ends, and a perforated section having the plurality of perforated irrigation tubes 36 extending outward between the second end and a third end).

Foam structure 46 may have a cylindrical geometry large enough to cover the entire area occupied by the plurality of perforated irrigation tubes 36 (or non-perforated, closed irrigation tubes 39 as depicted in FIG. 5B). For example, if each perforated irrigation tube 36 has a length of approximately 15 centimeters (approximately 6 inches) between first and second ends, then the foam structure 46 can have an equivalent diameter to embed each perforated irrigation tube 36 therein. This enables fluid dispersal out of the plurality of perforated irrigation tubes 36 and into the foam structure 46 for wound cleansing when used with second system 20. In other examples, foam structure 46 may have a cubical, pyramidal, pentagonal, hexagonal, or other suitable polygonal geometry as necessitated by the wound type or patient characteristics, for example. Foam structure 46 may have cutlines or perforations between irrigation tubes 36 to aid in cutting or tearing the foam into fingers for the purpose of fanning the irrigation tubes 36 over specific areas of a wound bed 104.

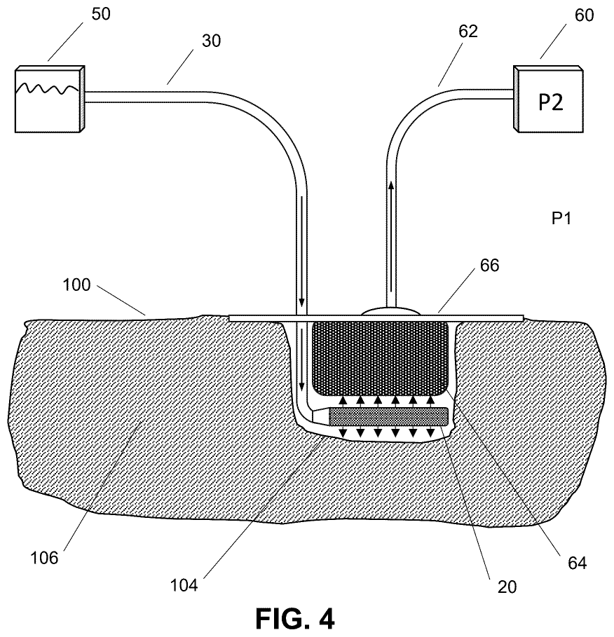

Referring now to FIG. 4, operation of the second wound irrigation system 20 in a large deep wound 104 of a patient is depicted. The second end of the irrigation tube 30 can be cut to length and coupled to a manifold 34 with a plurality of perforated irrigation tubes 36 extending outward from the second end (or non-perforated, closed irrigation tubes 39 as depicted in FIG. 5B). Manifold 34 and the perforated irrigation tubes 36 can be sized to fit the base of the wound bed 104. The plurality of perforated irrigation tubes 36 coupled to or embedded within a foam structure 46 can be inserted into the wound tunnel and positioned at the wound bed 104. The first end of the insertion tube 30 can be coupled or placed in fluid communication with an injection fluid source 50 configured to dispense or disperse fluid through the insertion tube 30. In other examples, a plurality of non-perforated, closed irrigation tubes 39 can be used in place or in addition to perforated irrigation tubes 36 (e.g., a combination of non-perforated and perforated irrigation tubes used simultaneously with second system 20).

A negative pressure wound therapy system can then be applied over the “irrigation device” comprising the irrigation tube 30, the manifold 34, the plurality of perforated irrigation tubes 36, and the foam structure 46 in combination. The negative pressure wound therapy system generally includes a layer of open-celled foam or other wound filler 64 sealed with a film or drape 66 and couplable via a negative pressure vacuum tube and wound dressing connector 62 to a negative pressure source 60. In examples, negative pressure source 60 may comprise a negative pressure pump with a pressure P₂ less than atmospheric pressure P₁.

After placement, the injection fluid source 50 can inject fluid through the irrigation device 30, 34, 36, 46 intermittently, continuously, or at a controlled rate into a patient wound. Fluid travels from the injection fluid source 50 to the first end of the irrigation tube 30, followed by traveling to the second end of the irrigation tube 30 and being dispersed out of the plurality of perforated irrigation tubes 36 (or non-perforated, closed irrigation tubes 39 as depicted in FIG. 5B) couplable to the second end via the manifold 34. The fluid can be dispersed into the foam structure 46 which the plurality of perforated irrigation tubes 36 are embedded within, followed by dispersal out of the foam structure 46 evenly onto and across the contours of the wound bed 104. Subsequently, the negative pressure source 60 can draw the fluid out of the wound bed 104 through the open-celled foam or other wound filler 64. The negative pressure vacuum tube 62 removes the cleansing fluid from the patient. This effectively removes contaminants and other debris from the wound which accelerates wound healing for the patient.

The entire operation of second wound irrigation system 20, or alternatively the “irrigation device” comprising irrigation tube 30, manifold 34, the plurality of perforated irrigation tubes 36 (or non-perforated, closed irrigation tubes 39 as depicted in FIG. 5B), and foam structure 46, is depicted in FIG. 4. Specifically, fluid is directed from the injection fluid source 50 through the irrigation tube 30 and the plurality of perforated irrigation tubes 36, followed by dispersal out of the foam structure 46 and onto the wound bed 104 defined within a patient skin surface 100 (e.g., the area defined between patient skin surface 100 and a length of patient soft tissue 106). The dispersed fluid can be drawn out of the wound bed 104 using the negative pressure source 60 in combination with the negative pressure vacuum tube 62 and the open-celled foam or other wound filler 64. Accordingly, second system 20 provides directed instillation or irrigation of fluid during wound therapy to deliver cleansing fluid precisely onto a wound bed to improve cleansing of larger deep, tunneling, undermined, and uneven wound beds, among other wound types.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A device for providing directed fluid irrigation to a wound, comprising:
an irrigation tube extending between a first end and a second end; and
a foam structure proximate the second end, wherein the foam structure is configured to dispense fluid received from the irrigation tube uniformly into a wound for cleansing.

2. The device of claim 1, wherein the foam structure comprises a hydrophobic, open-celled foam.

3. The device of claim 1, wherein the second end comprises a perforated section having a plurality of perforations arranged around an outer circumference of the second end.

4. The device of claim 3, wherein the plurality of perforations is arranged entirely around the outer circumference of the second end.

5. The device of claim 3, wherein the plurality of perforations is arranged partially around the outer circumference of the second end.

6. The device of claim 1, wherein the second end of comprises a section having a plurality of perforated or non-perforated irrigation tubes extending outward from the irrigation tube.

7. The device of claim 1, wherein the foam structure has one of a cylindrical geometry, a prismatic geometry, and a flat sheet geometry.

8. The device of claim 1, further comprising a manifold positioned between the second end and the foam structure, the manifold configured to distribute fluid from the irrigation tube to the foam structure for dispensing into a wound.

9. The device of claim 1, wherein the second end comprises a closed section terminating in the foam structure.

10. A system for providing directed fluid irrigation to a wound, comprising:
an irrigation device having:
an irrigation tube extending between a first end and a second end; and
a foam structure proximate the second end; and
an injection fluid source coupled to the first end of the irrigation tube, the injection fluid source configured to provide fluid to the irrigation tube between the first end and the second end,
wherein the foam structure is configured to dispense fluid received from the irrigation tube into a wound for cleansing.

11. The system of claim 10, further comprising a negative pressure system including:
a negative pressure source having a pressure P₂ less than an atmospheric pressure P₁;
a layer of wound filler; and
a vacuum tube connector coupled to the negative pressure source at a first end and coupled to the layer of wound filler at a second end,
wherein the negative pressure source is configured to remove fluid from a wound by drawing the fluid through the layer of wound filler and out through the vacuum tube connector.

12. The system of claim 10, wherein the foam structure comprises a hydrophobic, open-celled foam.

13. The system of claim 10, wherein the second end comprises a perforated section having a plurality of perforations arranged around an outer circumference of the second end.

14. The system of claim 10, wherein the second end comprises a section having a plurality of perforated or non-perforated irrigation tubes extending outward from the irrigation tube.

15. The system of claim 10, wherein the foam structure has one of a cylindrical geometry, a prismatic geometry, and a flat sheet geometry.

16. The system of claim 10, wherein the second end comprises a closed section terminating in the foam structure.

17. A method for providing directed fluid instillation or irrigation to a wound, comprising:
positioning an irrigation device proximate a wound, wherein the irrigation device comprises an irrigation tube extending between a first end and a second end, wherein the irrigation device comprises a foam structure proximate the second end; and
injecting cleansing fluid through the irrigation tube to the foam structure and outward from the foam structure to the wound for cleansing.

18. The method of claim 17, wherein the second end of the irrigation tube comprises one of:
a perforated section having a plurality of perforations arranged around an outer circumference of the second end;
a section having a plurality of perforated or non-perforated irrigation tubes extending outward from the irrigation tube; and
a closed section terminating in the foam structure.

19. The method of claim 18, further comprising trimming the irrigation tube and the foam structure to a size that fits a geometry of the wound.

20. The method of claim 17, further comprising applying negative pressure to the irrigation device to remove fluid and contaminants from the wound, wherein applying negative pressure to the irrigation device is performed using a negative pressure system comprising:
a negative pressure source having a pressure P₂ less than an atmospheric pressure P₁;
a layer of wound filler; and
a vacuum tube connector coupled to the negative pressure source at a first end and coupled to the layer of wound filler at a second end,
wherein the negative pressure source is configured to remove fluid from a wound by drawing the fluid through the layer of wound filler and out through the vacuum tube connector.
